(19) United States
(12) Patent Application Publication
(10) Pub. No.: US 2007/0113860 A1
(43) Pub. Date: May 24, 2007

(54) Tracking apparatus and a method of using

(76) Inventor: Peter Traneus Anderson, Andover, MA (US)

Correspondence Address: BERKELEY LAW & TECHNOLOGY GROUP, LLP, 1700 NW 167TH PLACE, SUITE 240, BEAVERTON, OR, 97006, US

(21) Appl. No.: 11/285,831

(22) Filed: Nov. 22, 2005

(51) Int. Cl. A61B 19/00

(52) U.S. Cl. 128/898

(57) ABSTRACT

Embodiments of methods, apparatuses, devices, and/or systems for performing image guided surgery are described. In one particular embodiment, a tracking apparatus may be employed to perform one or more aspects of image guided surgery.

### BACKGROUND

This disclosure is related to tracking apparatuses, methods and systems, such as may be employed in image guided surgery procedures.

Image guided surgery may provide surgeons with access to particular information during a surgical procedure, which may enable less invasive procedures, for example. In at least one type of image guided surgery, images of a patient may be obtained either prior to surgery or intra-operatively. During the procedure, the position and/or orientation of one or more surgical instruments may be tracked and/or marked, such as with respect to the obtained images, for example.

One difficulty with state of the art technology for image guided surgery is concerning the devices utilized as part of the procedure. For example, devices may be complex, expensive and/or cumbersome to use. For a variety of reasons, it may be desirable to develop devices that address one or more of these limitations.

### BRIEF DESCRIPTION OF THE DRAWINGS

Subject matter is particularly pointed out and distinctly claimed in the concluding portion of the specification. Claimed subject matter, however, both as to organization and method of operation, together with objects, features, and advantages thereof, may best be understood by reference of the following detailed description when read with the accompanying drawings in which:

FIG. 1 is a block diagram of one embodiment of a tracking apparatus;

FIG. 2 is a block diagram of one embodiment of a tracking apparatus;

FIG. 3 is a cut-away view of a portion of a tracking apparatus;

FIG. 4 is a block diagram of one embodiment of an image guided surgery system; and

FIG. 5 is a flowchart illustrating one embodiment of method of using a tracking apparatus.

### DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, well-known methods, procedures, components and/or circuits have not been described in detail so as not to obscure claimed subject matter.

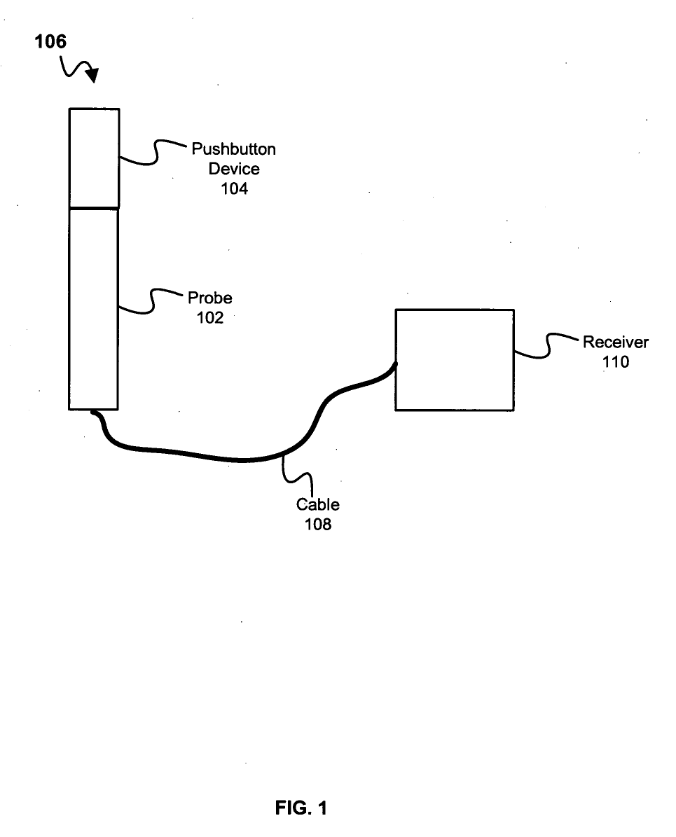

As pointed out previously, one limitation with state of the art technology may be that devices utilized as part of a surgical procedure, such as a tracking and/or marking device, may be expensive, complex and/or cumbersome to use. Referring now to FIG. 1, there is illustrated an assembly 106 that may be utilized either prior to surgery or intra-operatively. Assembly 106 may include a probe 102. Probe 102 may provide a tracking device, for use prior to and/or during a surgical procedure, for example. The position and/or orientation of the probe 102 may be determined, such as continuously or periodically during a procedure, and as a result probe 102 may allow a surgeon to accurately perform surgical tasks, for example. A device such as probe 102 may obtain positioning information to provide additional information to a surgeon during a surgical procedure and/or to generate an image that may be utilized as part of a procedure, for example. Probe 102 may be tracked during at least a portion of a surgical procedure. Additionally, assembly 106 may comprise a pushbutton device 104. Pushbutton device 104 may be capable of being placed in a pushed state and/or a not pushed state. In one example, pushbutton device 104 may be placed in a pushed state, such as during a surgical procedure. The position of the probe may be recorded in response to pushbutton device being placed in a pushed state, at some time proximate to the time the pushbutton is placed in a pushed state. The position may be recorded, or marked, by a device in communication with the assembly 106, such as receiver 110 and/or additional circuitry not shown in detail, for example.

Probe 102 may be in communication with receiver 110, and may be capable of sending and/or receiving signals. Signals may be provided to receiver 110 by use of cable 108, in at least one embodiment. Receiver 110 may include receiving circuitry and/or may be coupled to processing circuitry such as a computer system (not shown). In operation, probe 102 may provide signals to receiver 110 by use of cable 108, and as a result receiver 110 and/or processing circuitry may be capable of processing the signals to substantially determine one or more parameters of probe 102, such as the position and/or orientation of probe 102. Additionally, pushbutton device 104 may provide a signal to receiver 110 by use of cable 108, such as when placed in a pushed state. This may result in receiver 110 detecting the pushing of pushbutton device 104 and/or marking the position of probe 102 at the time pushbutton device 104 is pushed, for example.

Assembly 106 may utilize wired communications media such as cable 108 to communicate with receiver 110 or, alternatively, may employ wireless signals to communicate with receiver 110. For example, although not illustrated in detail, probe 102 and/or the receiver 110 may employ one or more coils as transmitters and/or receivers. Such coils may be adapted to generate electromagnetic signals and transmit the electromagnetic signals wirelessly to the receiver 110, in response to a current from a power source. Probe 102 and receiver 110 may employ an Industry Standard Coil Architecture (ISCA), utilizing three orthogonally oriented transmitter and receiver coils. In operation, such three transmitter coils may be capable of generating wireless signals which may be detected by the receiver coils, such as by measuring the mutual inductance between the transmitter and/or receiver coils. The generated wireless signal transmitted from the three transmitter coils may be processed to determine a position and orientation of the transmitter coils with respect to the three receiver coils. Additionally, although not illustrated in detail, pushbutton 104 may comprise a permanent magnet (not shown) mounted proximate to a Hall-effect sensor. When pushbutton 104 is placed in a pushed state, the magnet may be moved toward the Hall-effect sensor, and result in the generation of a wire-line signal which may be transmitted to the receiver by use of cable 108, for example.

Assembly 106 may be utilized during a surgical procedure and, depending on the procedure and/or particular use, assemblies such as assembly 106 may comprise a single use assembly that may be disposed of after the procedure. However, assembly 106 may be expensive and/or complex, and disposing of an assembly such as assembly 106 may not be economically feasible. Additionally, use of a communication media such as a cable 108 may be cumbersome, such as by restricting the range of motion of assembly 106, for example. As alluded to previously, it may be desirable to develop an assembly comprising a probe and a pushbutton device that may be less expensive and/or less cumbersome, for example. Such an assembly may be illustrated in the accompanying figures and explained in more detail herein.

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” and/or “an embodiment” in various places throughout this specification are not necessarily all referring to the same embodiment. Furthermore, the particular features, structures, and/or characteristics may be combined in one or more embodiments.

“Circuitry” as referred to herein relates to structure adapted to perform one or more logical operations. For example, circuitry may be adapted to provide one or more output signals based at least in part on one or more input signals. Such circuitry may provide a digital output signal, or circuitry which provides one or more analog output signals in response to one or more analog input signals. Such circuitry may be provided, for example, in an application specific integrated circuit (ASIC) and/or a field programmable gate array (FPGA). Also, circuitry may utilize machine-readable instructions stored in a storage medium in combination with a processor or other processing circuitry to execute such machine-readable instructions. However, these are merely examples of structures which may perform on or more operations, and claimed subject matter is not limited in these respects.

Unless specifically stated otherwise, as apparent from the following discussion, it is appreciated that throughout this specification discussions utilizing terms such as “processing,” “computing,” “calculating,” “selecting,” “forming,” “obtaining,” “modifying,” “sending,” “receiving,” “transmitting,” “storing,” “determining” and/or the like refer to the actions and/or processes that may be performed by a computing platform, such as a computer or a similar electronic computing device, that manipulates and/or transforms data represented as physical, electronic and/or magnetic quantities and/or other physical quantities within the computing platform's processors, memories, registers, and/or other information storage, transmission, reception and/or display devices. Accordingly, a computing platform refers to a system or a device that includes the ability to process and/or store data in the form of signals. Thus, a computing platform, in this context, may comprise hardware, software, firmware and/or any combination thereof. Further, unless specifically stated otherwise, a process as described herein, with reference to flow diagrams or otherwise, may also be executed and/or controlled, in whole or in part, by a computing platform.

A surgical procedure may be performed on a surgical object, and a surgical object may comprise a patient or a portion thereof, for example. Surgical procedures may be performed by a user, and by use of one or more surgical instruments. Precision placement and/or movement of the one or more surgical instruments may be important, particularly if the surgical object is difficult to see or located internally to a patient, for example. Image-guided surgery is a surgical procedure wherein one or more images may be provided to a surgeon that may be generated prior to and/or during a surgical procedure. The images may represent one position, orientation or placement of a patient and/or from a surgical instrument. Images such as these may provide a surgeon with the capability to navigate during a procedure and determine when a surgical instrument is in a desired location. Image guided surgery may be useful if a surgical object is difficult to see and/or located internally to a patient. Additionally, image guided surgery may allow for less invasive procedures and/or enhanced control of surgical instruments by providing position and/or orientation of a surgical instrument that may be used internally with respect to a patient. Enhanced control may result in reduced risk during a surgical procedure. Additionally, it is worthwhile to note that in this context, a surgeon may refer to a human surgeon, but may additionally refer to a robotic surgeon, such as a device capable of performing surgical procedures automatically, and/or a device that may be at least partially controlled by a human surgeon.

One particular type of image-guided surgery may utilize surgical instrument parameters, such as orientation and/or position data. Instrument parameters may include orientation and/or position data of an instrument, and/or may comprise historical orientation and/or position data. Historical orientation and/or position data may also be referred to as tracking data, and may be determined based on one or more coordinate systems. Instrument data may include, relative x, y, and/or z position of the instrument, and/or pitch, yaw and/or roll of the instrument, as just a few examples. For a variety of reasons, it may be desirable to provide the capability to mark and/or record positions on a surgical object, by use of a switch, such as a pushbutton that may be coupled with the surgical instrument. Additionally, it may be desirable to provide a less complex and/or less cumbersome apparatus and method of tracking and/or marking the position of an instrument such as during a surgical procedure. However, as explained previously, state of the art tracking instruments may not address one or more of these limitations.

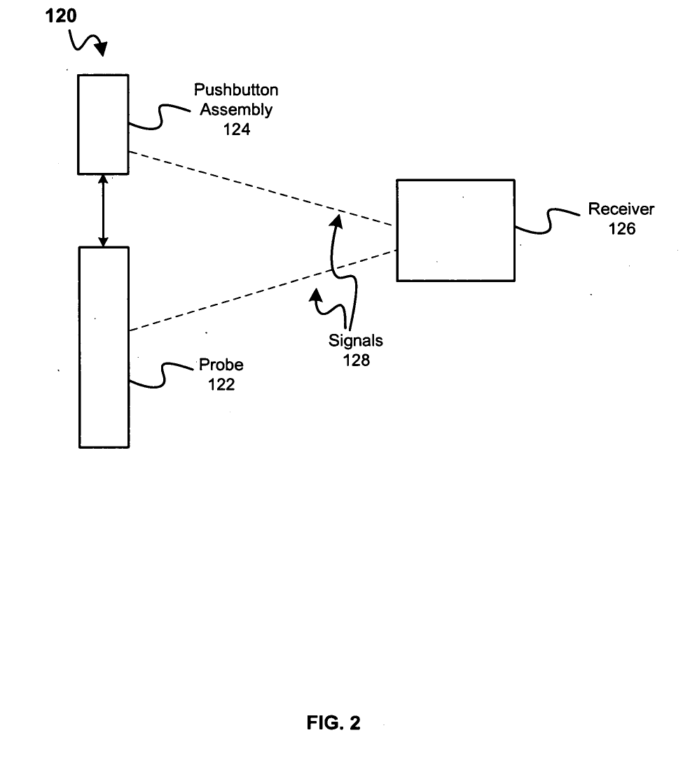

FIG. 2 is a schematic diagram of a surgical instrument 120 that may be utilized as part of a surgical procedure. In one embodiment, surgical instrument 120 may comprise a probe 122. However, it is worthwhile to note that claimed subject matter is not so limited. For example, in alternative embodiments, surgical instrument 120 may comprise an anatomical device, a probe, a drill, a guide, a catheter, a stimulator, a debrider, an aspirator, a curette, forceps, a microscope, an endoscope, and/or one or more implants, as just a few examples. Surgical instrument 120 may include electronics (not shown), that may be integrated and/or adjunct to the instrument. The electronics may be adapted to transmit, generate and/or provide one or more wireless and/or wire-line signals 128 to a receiver 126. The signals 128 may be received and/or processed by circuitry that may be implemented as part of a computing system (not shown). The signals may be utilized, at least in part, to substantially determine the position and/or orientation of surgical instrument 120, and/or to indicate that the position and/or orientation of the instrument should be recorded, for example.

Surgical instrument 120 may include electronics (not shown) that may be adapted to generate wireless signals 128, which may be detected and/or received by receiving electronics. As mentioned previously, it may be desirable to provide tracking capabilities for a surgical instrument. Wireless signals 128 may be generated by surgical instrument 120, and received by a receiving device such as receiver 126. The instrument 120 may comprise a probe 122, as mentioned previously. The probe 122 may include a transmitter portion (not shown) that may include electronics such as one or more transmitter coils. Although the particular configuration of the transmitter portion is not limited, in one embodiment the transmitter portion may be configured in an ISCA configuration which, as described previously, may employ three transmitter coils oriented orthogonally with respect to one another. Alternatively, in at least one embodiment, the transmitter portion may comprise a single transmitter coil. The single transmitter coil may be capable of generating a wireless signal 128 in response to a power source. The coil may be driven at a certain frequency to emit electromagnetic signals which may be provided wirelessly to a receiver 126. Although wireless signal 128 may comprise an electromagnetic signal, the claimed subject matter is not so limited, and may comprise other types of signals that may provide data, such as optical signals, for example. The receiver 126 may include circuitry capable of detecting signal 128 and/or processing signal 128 to substantially determine a position of the probe 122 based at least in part on information in the signal 128, for example.

Although claimed subject matter is not limited to any particular type of transmitter and/or receiver configuration, one potential embodiment of a wireless transmitter is disclosed in U.S. patent application Ser. No. 10/611,112, assigned to the assignee of the presently claimed subject matter. For example, a transmitter may comprise a single transmitter coil, and may be capable of emitting an electromagnetic signal. Receiver 126 may comprise circuitry capable of detecting and/or processing signal 128 to substantially determine transmitter position and/or orientation. In one embodiment, receiver 126 may comprise a plurality of coils (not shown). Such coils may receive and process wireless signals and provide the processed signals to additional circuitry. The additional circuitry may be adapted to analyze and/or process the received signal and/or additional properties, such as to determine one or more characteristics. For example, in operation, coils of a transmitter may be provided with a current. The transmitter may generate electromagnetic signals, which may induce voltages in the coils of a receiver. A mutual inductance between the transmitter and receiver coils may be determined, and may be utilized to substantially determine position of the transmitter, for example. However, it is worthwhile to note that the particular configuration of a transmitter, a receiver and/or processing circuitry is not limited and claimed subject matter may include devices and/or techniques for transmitting, receiving and processing either now known or later discovered.

It may be desirable not only to track a surgical instrument, but also to mark and/or record particular position data for such a surgical instrument. This may be performed, in one embodiment, by sending a wireless signal 128 to receiving device 126 at a particular time when recording and/or tracking may be desirable. Signal 128 may indicate that a current position of an instrument should be determined and/or recorded, for example. In this embodiment, surgical instrument 120 may comprise a pushbutton assembly 124. However, it is worthwhile to note that the claimed subject matter is not limited to use of a pushbutton. For example, surgical instrument 120 may implement additional types of a switch that may be activatable, meaning, in this context, that the switch may have at least two states, an activated state and an inactivated state, for example. Pushbutton assembly 124 may be removably and/or permanently coupled to probe 122 and/or may be integrated with the probe, for example. Pushbutton assembly 124 may be activatable to generate signal 128. Signal 128 may comprise a wireless signal, and may be receivable by receiver 126. Signal 128 may be generated when the button is activated, which may comprise placing the button of pushbutton assembly 124 in a pushed state, such as by applying pressure to the pushbutton. However, this is merely an example of how a signal may be generated in response to a manual action and claimed subject matter is not so limited. This may provide the capability of a receiver 126 to receive a signal and determine whether a pushbutton is placed in a pushed or not pushed state, based at least in part on the signal.

Additionally, pushbutton assembly 124 may allow receiver 126 and/or other circuitry (not shown) such as processing circuitry that may be at least partially embodied in a computing system (not shown) to track the position of the surgical instrument 120 and/or record particular locations of a surgical object (not shown) when pushbutton assembly 124 is placed in a pushed and/or not pushed state. For example, probe 122 may include a transmitter portion capable of generating a first signal. The first signal may comprise a wireless signal that may be generated by one or more coils (not shown) implemented as part of the transmitter. The first signal may comprise an electromagnetic signal, in at least one embodiment. The first signal may be processed by receiver 126 and/or additional circuitry to substantially determine position and/or orientation of the surgical instrument, for example. Additionally, pushbutton assembly 124 may be capable of generating a second signal. The second signal may comprise a wireless signal, and may be distinguishable from the first signal, such as by having a differing frequency, for example. The second signal may comprise an electromagnetic signal, in at least one embodiment. The generated second signal may be transmitted to receiver 126, and receiver 126 and/or processing circuitry may utilize the signal to substantially determine whether pushbutton assembly 124 is in a pushed state, for example. In operation, when pushbutton assembly 124 is in a pushed state, the generated signal may be received by receiving circuitry of receiver 126. The receiving circuitry and/or processing circuitry may determine that the button was pushed, and, in response may record the position of the surgical instrument. The position of the surgical instrument may be determined based at least in part on the first signal received from a transmitter, for example.

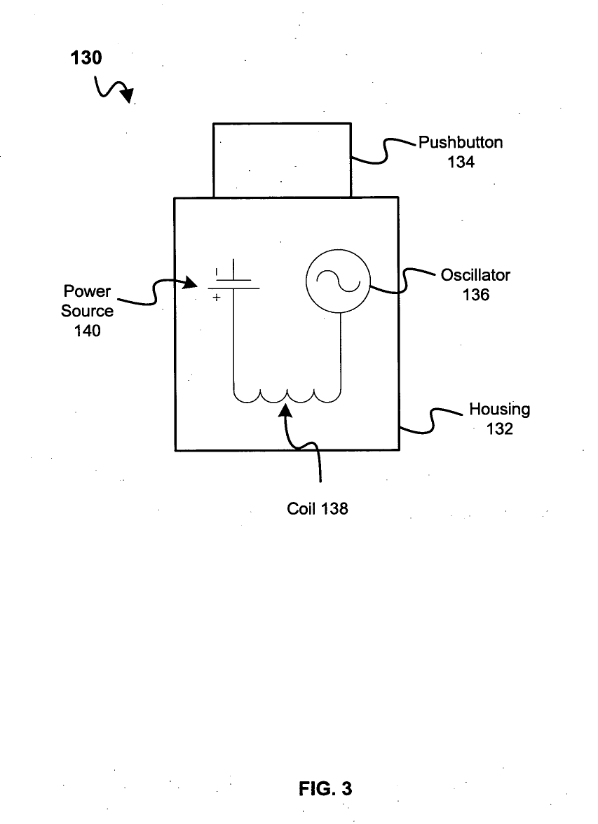

Surgical instrument 120 may be formed in a variety of ways and by use of a variety of materials. However, in one embodiment a surgical instrument may comprise a housing to enclose components of the surgical instrument as described previously. The housing may be formed from a variety of materials, such as plastics and/or synthetic materials that may include properties such as sterility and/or low cost, as just a few examples. Additionally, the housing may be formed from materials that may not interfere with generated magnetic fields, such as non-ferrous materials, for example. Although portions of surgical instrument 120, such as pushbutton assembly 124 may comprise a variety of types and/or configurations, one embodiment may be better understood with reference to FIG. 3.

Referring now to FIG. 3, there is illustrated a cut-away view of a pushbutton assembly 130 that may be employed as part of a surgical instrument. Pushbutton assembly 130 may include a housing 132 coupled to a pushbutton 134. Pushbutton assembly 130 may be adapted to be removably or permanently coupled to a device such as a probe. Pushbutton 134 may comprise a normally open pushbutton 134 or, in other words may comprise a pushbutton 134 in a normally not pushed state, for example, and may be employed in circuitry as illustrated. Although the claimed subject matter is not so limited, in this embodiment pushbutton 134 may be formed as part of a circuit as illustrated, and may be coupled in series with a power source 140 and an oscillator 136, for example. Power source 140 may comprise a battery capable of providing a direct current power signal, although alternative power sources may include photocells and/or radio frequency energy as just a few alternatives. In this configuration, when pushbutton 134 is in a pushed state, power source 140 may be coupled to the oscillator 136, and oscillator 136 may drive a coil 138 to generate an electromagnetic signal having a frequency, for example. The particular frequency may depend at least in part on the design and/or configuration of the oscillator circuitry and one or more components of the circuitry, and it is worthwhile to note that claimed subject matter is not limited in this respect. For example, pushbutton assembly 130 may include any configuration that may provide an activatable switch, that, when activated may generate a signal that may be detectable by a receiving device, for example.

In this embodiment, when pushbutton 134 is in a pushed state, in at least one embodiment, an electromagnetic signal having a frequency may be generated. The electronic signal may be generated when pushbutton 134 closes the circuit and power is provided to the oscillator 136. When powered, oscillator 136 may drive coil 138 to generate an electromagnetic signal having a frequency, for example. The electromagnetic signal may be transmitted wirelessly and detected by a receiver (not shown), and may indicate that the position of a surgical instrument should be tracked and/or recorded. The position and/or orientation may be determined based on another electromagnetic signal, such as a wireless signal generated by a transmitter, as explained previously. This may provide the capability of a surgeon to track and/or determine the position of a surgical instrument by pressing button 132, for example. The electromagnetic signal may be provided to a receiver that is additionally configured to detect one or more electromagnetic signals generated by a transmitter, as explained previously. The detected signals may be provided to processing circuitry to substantially determine whether a button is pushed.

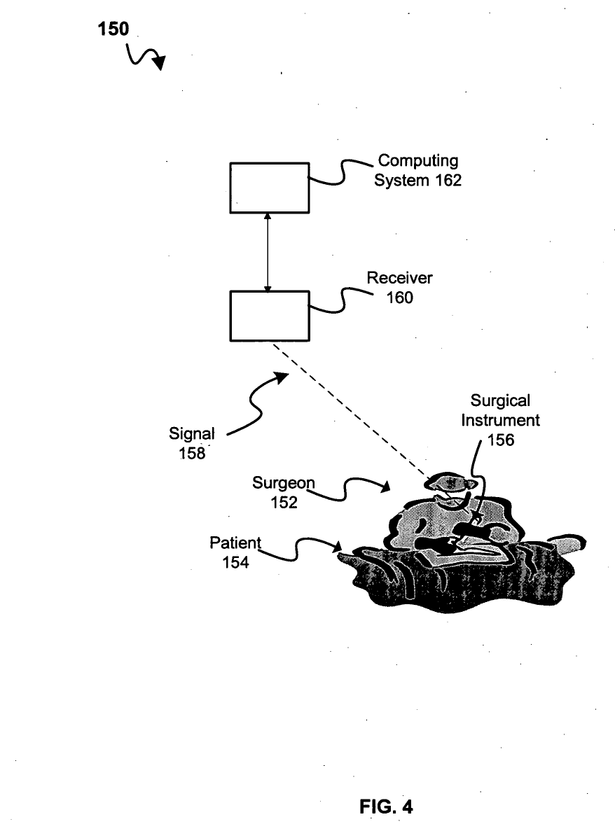

The pushbutton assembly 130 may be employed as part of a surgical instrument such as surgical instrument 120 of FIG. 2. In at least one embodiment, the pushbutton assembly 130 may be removably coupled with the transmitter portion of surgical instrument 120, such as by a snap receiver (not shown). In this embodiment, pushbutton assembly 130 may be capable of generating a wireless signal having a frequency that may be distinguishable from a wireless signal generated by a transmitter. For example, such a transmitter may be capable of generating a first wireless signal having a first frequency, and the pushbutton may be capable of generating a second wireless signal having a second frequency. The first and second wireless signals may be received by a receiver. The receiver and/or processing circuitry may be capable of detecting and/or distinguishing the first and second wireless signals. For example, the receiver and/or processing circuitry may be adapted to receive a first and a second wireless signal from a transmitter and a pushbutton assembly, respectively. The receiver and/or processing circuitry may be adapted to process the signals and substantially determine position and/or orientation of the transmitter and/or pushbutton assembly, and/or may be adapted to determine whether a pushbutton of the pushbutton assembly has been placed in a pushed or not pushed state, for example. Utilization of a pushbutton assembly as illustrated in FIG. 3 is explained in more detail with reference to FIG. 4.

FIG. 4 illustrates a block diagram of a system 150 employing a surgical instrument 156. System 150 may include a surgeon 152 performing a surgical procedure on a patient 154 and/or a surgical object. In this embodiment, although not shown in detail, surgical instrument 156 may include a transmitter and/or a pushbutton assembly. For example, surgical instrument 156 may include a transmitter portion capable of generating a first signal, and a pushbutton assembly portion capable of generating a second signal, as illustrated above according to a particular embodiment. The first and/or second signal may comprise wireless signals, and may have differing and/or distinguishable frequencies, for example. Surgical instrument 156 may comprise one or more types of surgical instrument, such as described previously. System 150 may include a receiver 160 that may be adapted to receive one or more signals generated by surgical instrument 156. In one embodiment receiver 160 may include a plurality of coils (not shown) adapted to detect and/or receive wireless signals such as electromagnetic signals. In a particular embodiment, receiver 160 may comprise a printed circuit board (PCB) and a plurality of coils (not shown). The plurality of coils may be arranged in some configuration to allow for the detection of wireless signals. The coils may be arranged in one or more arrays, for example, and/or may be electrically coupled to one another, for example.

System 150 may include a computing system 162 adapted to process the received signals. Computing system 162 may be adapted to process one or more signals, such as first and second signals that may be received by the transmitter portion and pushbutton assembly portion, respectively. Computing system 162 may be adapted to distinguish among the signals and/or determine one or more characteristics of the signals, and may be capable of determining, for example, position and/or orientation of the surgical instrument 156, and/or whether a pushbutton of the pushbutton assembly has been placed in a pushed or not pushed state.

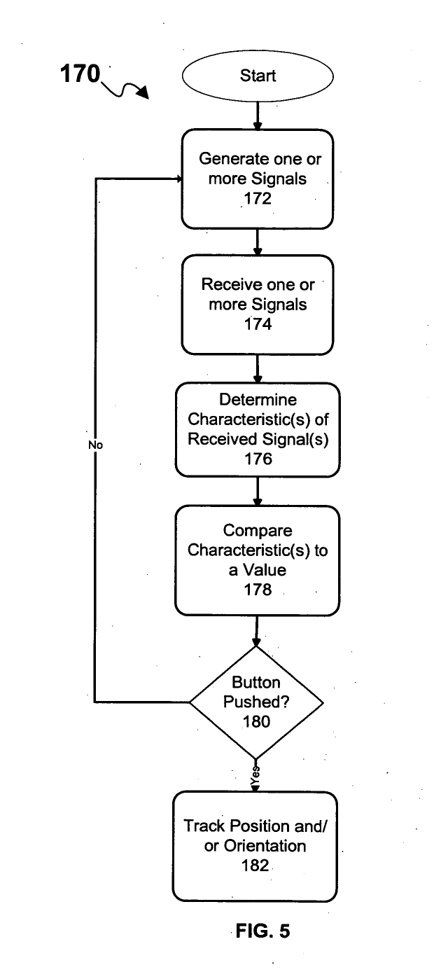

In operation, system 150 may be utilized in the following manner: A surgical instrument 156 may be utilized as part of a surgical procedure. The surgical instrument may include a transmitter portion that may periodically generate a wireless signal. The wireless signal may comprise an electromagnetic signal having a first frequency. Receiving circuitry and processing circuitry may be adapted to detect and/or process the wireless signal, and may additionally be capable of determining one or more characteristics of the surgical instrument 156, such as position and/or orientation of the instrument, based at least in part on the wireless signal. Surgical instrument 156 may additionally include a pushbutton assembly. A user, such as surgeon 152, may place the pushbutton in a pushed state when the surgical instrument is in a position and/or orientation that the user may desire to record. For example, a user may desire to record a location of a surgical object, and may push the button to record the position. Pushing the button may result in the pushbutton assembly generating a wireless signal, such as an electromagnetic signal. The wireless signal may have a second frequency differing from the frequency of the wireless signal generated by the transmitter. The receiver circuitry and/or processing circuitry may receive the wireless signal generated by the pushbutton assembly. The receiver circuitry and/or processing circuitry may determine the state of pushbutton by analyzing the received wireless signal and determining one or more characteristics of the signal, for example. If a determination is made that the pushbutton is pushed, the particular position and/or orientation of the surgical instrument may be recorded. However, claimed subject matter is not so limited, and system 150 may additionally be employed in a manner as described with reference to FIG. 5.

FIG. 5 is a flow diagram of an embodiment 170 of a method of using a tracking apparatus, such as a surgical instrument. However, claimed subject matter is not limited in scope to this particular example. For example, for flow diagrams presented herein, the order in which blocks are presented does not necessarily limit claimed subject matter to any particular order. Additionally, intervening blocks not shown may be employed without departing from the scope of claimed subject matter. Likewise, flow diagrams depicted herein may, in alternative embodiments, be implemented as a combination of hardware, software and/or firmware, such as part of a computer or computing system.

One or more signals may be generated at block 172. The one or more signals may comprise wireless and/or electromagnetic signals, although other signals, such as optical signals may be utilized in accordance with alternative embodiments. The one or more signals may be generated by a surgical instrument. The instrument may comprise a probe, as mentioned previously. The probe may include a transmitter portion that may include electronics such as one or more transmitter coils. The transmitter may comprise one or more configurations, such as the aforementioned ISCA configuration or single transmitter coil configuration. The transmitter may be capable of emitting an electromagnetic signal, by driving the one or more coils at a certain frequency to emit a signal. Additionally, the instrument may comprise a pushbutton assembly activatable to generate a signal. The signal may comprise an electromagnetic signal that may be generated by a coil driven by an oscillator, as explained previously. The surgical instrument may be adapted to generate a first and a second signal. The first signal may have a first frequency, and may be utilized to substantially determine position and/or orientation of the surgical instrument. The second signal may have a second frequency, and may be utilized to substantially determine whether the pushbutton is pushed, for example.

At block 174, the one or more generated signals may be received by a receiver. The receiver may comprise receiving circuitry, and may be adapted to receive signals having one or more frequencies. Additionally, the receiver and/or processing circuitry may be adapted to determine one or more characteristics of the received signals. In one embodiment, a first and a second signal may be generated and received. At block 176 one or more characteristics of the first and/or second received signal may be determined. For example, the receiving and/or processing circuitry may be capable of measuring the magnitude of one or more of the signals and/or may be capable of determining the sum of the squares of the magnitude. However, claimed subject matter is not limited to these characteristics. For example, any characteristics that may be determined for a wireless signal may be utilized in at least one embodiment. The characteristics may be measured and/or calculated based at least in part on the wireless signal, for example. However, in one embodiment, the sum of the squares of the magnitude of the second signal may be determined. This value may be determined by measuring one or more coils of a receiver, such as a receiver implementing multiple coils, and performing one or more calculations by use of processing circuitry, for example. After determining one or more characteristics, a comparison may be performed at block 178.

At block 178, one or more characteristics of the received signal may be compared to a value. In one embodiment, the value may comprise a threshold value. The comparison may be substantially performed to determine the state of a pushbutton of a pushbutton that may be pushed at block 180. For example, when a pushbutton such as pushbutton assembly 130 is placed in a pushed state, a wireless signal may be generated, as explained previously. The wireless signal may comprise an electromagnetic signal, and may be received by receiving circuitry. The receiving circuitry may comprise one or more coils. The one or more coils, along with processing circuitry, may be capable of determining one or more characteristics of the electromagnetic signal, such as a sum of the squares of the magnitude of the signal, although, again, additional characteristics may be utilized in alternative embodiments. The one or more characteristics may be compared with a threshold value. In one example, a signal characteristic exceeding a threshold value may indicate that the button is pushed. For example, a sum of the squares of the magnitude of the received signal may be compared to a threshold value that may be determined based on the circuitry of a pushbutton assembly and/or a surgical instrument. Conversely, if the button is not pushed, the measurement may not exceed a threshold value. In this embodiment, if the threshold value is exceeded by the signal measurement, at block 182, position and/or orientation of the surgical instrument may be tracked. Tracking may be performed by utilizing one or more transmitter signals, such as the first signal. The transmitter signals may be utilized in a manner described previously in connection with particular embodiments. Additionally, it is worthwhile to note that in one or more embodiments, one or more characteristics of a wireless signal may be utilized to determine whether a pushbutton device is placed in a pushed and/or not pushed state in a manner not described in detail. The particular characteristics of a wireless signal as well as the particular utilization of the characteristics may depend at least in part on the circuitry and/or configuration of a pushbutton assembly and/or receiving circuitry, and the claimed subject matter is not limited to the embodiments described herein. Additionally, one or more portions of the flow diagram may be repeated, such as on a continual or periodic basis.

The following discussion details several possible embodiments for accomplishing this, although these are merely examples and are not intended to limit scope of claimed subject matter. As another example, one embodiment may be in hardware, such as implemented to operate on a device or combination of devices, for example, whereas another embodiment may be in software. Likewise, an embodiment may be implemented in firmware, or as any combination of hardware, software, and/or firmware, for example. Likewise, although claimed subject matter is not limited in scope in this respect, one embodiment may comprise one or more articles, such as a storage medium or storage media. This storage media, such as, one or more CD-ROMs and/or disks, for example, may have stored thereon instructions, that when executed by a system, such as a computer system, computing platform, or other system, for example, may result in an embodiment of a method in accordance with claimed subject matter being executed, such as one of the embodiments previously described, for example. As one potential example, a computing platform may include one or more processing units or processors, one or more input/output devices, such as a display, a keyboard and/or a mouse, and/or one or more memories, such as static random access memory, dynamic random access memory, flash memory, and/or a hard drive, although, again, claimed subject matter is not limited in scope to this example. It will, of course, be understood that, although particular embodiments have just been described, claimed subject matter is not limited in scope to a particular embodiment or implementation.

In the preceding description, various aspects of claimed subject matter have been described. For purposes of explanation, systems and configurations were set forth to provide a thorough understanding of claimed subject matter. However, it should be apparent to one skilled in the art having the benefit of this disclosure that claimed subject matter may be practiced without the specific details. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all such modifications and/or changes as fall within the true spirit of claimed subject matter.

1. A method, comprising:
wirelessly receiving at least one signal from a surgical instrument;
determining one or more characteristics of the received electromagnetic signal;
comparing at least a portion of the determined characteristics to a value; and
recording one or more parameters of the surgical instrument based at least in part on the comparison.

2. The method of claim 1, wherein the one or more parameters comprise position and/or orientation of the surgical instrument.

3. The method of claim 1, wherein the determined characteristics comprise the sum of the squares of the magnitude of the received signal.

4. The method of claim 3, and further comprising:
recording the position and/or orientation of the surgical instrument by use of processing circuitry, if the sum of the squares of the magnitude exceeds the value.

5. The method of claim 4, wherein the position and/or orientation are substantially determined from data included in one or more of the electromagnetic signals.

6. The method of claim 1, wherein the at least one signal is wirelessly transmitted by one or more coils coupled to the surgical instrument.

7. The method of claim 6, wherein the position and/or orientation data is substantially determined by measuring a mutual inductance between the one or more coils and one or more additional coils located remote from said one or more coils.

8. The method of claim 1, and further comprising wirelessly receiving a plurality of signals, wherein at least a portion of the received signals comprise different electromagnetic frequencies.

9. The method of claim 6, wherein the at least one signal is generated in response to user activation of the one or more coils coupled to the surgical instrument.

10. An apparatus, comprising:
a surgical instrument, the surgical instrument having a plurality of wireless signal generating portions, wherein the wireless signal generating portions are capable of wirelessly transmitting a first and a second wireless signal having differing electromagnetic frequencies.

11. The apparatus of claim 10, and further comprising two signal generating portions, wherein at least one of the signal generating portions is adapted to wirelessly transmit a signal in response to user activation.

12. The apparatus of claim 11, wherein user activation is substantially performed by use of a pushbutton.

13. The apparatus of claim 10, wherein at least one of the signal generating portions comprises a single coil.

14. The apparatus of claim 10, wherein said surgical instrument comprises one or more of: an anatomical device, a probe, a drill, a guide, a catheter, a stimulator, a debrider, an aspirator, a curette, forceps, a microscope, an endoscope, and/or one or more implants.

15. A system, comprising:
a surgical instrument comprising a switch, wherein switch includes an activatable portion and a wireless signal generating portion, wherein the wireless signal generating portion is adapted to generate a first wireless signal in response to activation of the switch; and
receiving circuitry adapted to receive at least a portion of the generated wireless signal.

16. The system of claim 15, and further comprising processing circuitry adapted to determine one or more characteristics of the received first wireless signal.

17. The system of claim 15, wherein the surgical instrument further comprises a transmitter portion adapted to wirelessly transmit a second wireless signal, wherein the first and the second wireless signals have distinguishable electromagnetic frequencies.

18. The system of claim 15, wherein the switch comprises a pushbutton assembly.

19. The system of claim 16, wherein the determined characteristics comprise the sum of the squares of the magnitude of the first wireless signal.

20. The system of claim 16, wherein the receiving circuitry is further adapted to determine a position and/or orientation of the surgical instrument based at least in part on the determined characteristics.

21. The system of claim 20, wherein the position and/or orientation data is substantially determined by measuring a mutual inductance between one or more coils of the receiving circuitry and one or more coils of the transmitter portion.

22. The system of claim 17, wherein the transmitter portion comprises a plurality of coils arranged in an Industry Standard Coil Architecture (ISCA) configuration.

23. The system of claim 17, wherein the wireless pushbutton assembly and the transmitter portion are removably coupled by use of a snap receiver.
